(19) United States
(12) Patent Application Publication
(10) Pub. No.: US 2022/0047665 A1
(43) Pub. Date: Feb. 17, 2022

(54) PHYTONUTRIENT COMPOSITIONS ABLE TO LOWER POSTPRANDIAL BLOOD-GLUCOSE LEVELS THROUGH REGULATION OF SIGNAL TRANSDUCTION CASCADES

(71) Applicant: Brilliant Lab LLC, American Fork, UT (US)

(72) Inventors: Daniel A. Gubler, Orem, UT (US); Jeremy Tsang, Vineyard, UT (US)

(21) Appl. No.: 17/401,897

(22) Filed: Aug. 13, 2021

Related U.S. Application Data
(60) Provisional application No. 63/066,034, filed on Aug. 14, 2020.

(51) Int. Cl. A61K 36/42; A61K 31/353; A61K 36/88; A61K 36/79; A61K 36/24; A61K 31/7048; A61K 36/54; A61K 33/30; A61K 31/12; A61K 31/704; A61K 45/06

(52) U.S. Cl. CPC A61K 36/42; A61K 31/353; A61K 36/88; A61K 36/79; A61K 36/24; A61K 45/06; A61K 36/54; A61K 33/30; A61K 31/12; A61K 31/704; A61K 31/7048

(57) ABSTRACT

Described, among other things, are dosage form for administration to a subject, the dosage forms having at least four ingredients selected from okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus. Upon administration to a mammalian subject, the dosage forms generally promote the subject's health through the maintenance of a healthy blood sugar level.

### CROSS-REFERENCE TO RELATED APPLICATION

This application claims the benefit under 35 U.S.C. § 119(e) of U.S. Provisional Patent Application Ser. No. 63/066,034, filed Aug. 14, 2020, the disclosure of which is hereby incorporated herein in its entirety by this reference.

### TECHNICAL FIELD

The application relates generally to nutritional supplements and associated methods of making and using them. More particularly, the application relates to compositions able to lower postprandial blood-glucose levels in a subject administered the composition.

### BACKGROUND

Human metabolic health is reaching a crisis point. One in three Americans have metabolic syndrome. Moore et al. “Metabolic Syndrome Prevalence by Race/Ethnicity and Sex in the United States, National Health and Nutrition Examination Survey,” 1988-2012. Prev Chronic Dis 2017; 14: 160287. DOI: http://dx.doi.org/10.5888/pcd14.160287. 10.5% of the U.S. population is diabetic—with a large number of Americans undiagnosed. (available on the web at diabetes.org/resources/statistics/statistics-about-diabetes#:˜:text=Prevalence%3A%20In%202018%2C%2034.2%20million,of%20the%20population%2C%20had%20diabetes) 34.5% of adults in the U.S. have prediabetes meaning that almost 50% of the country's population is either diabetic or pre-diabetic.

The total cost of diabetes in the U.S., as of 2017, was estimated to be $327 billion. Pharmaceutical treatments, such as metformin, can lower blood-glucose levels via a mono-pharmacology model, but can have negative side effects. See, e.g., Siavash et al. “Severity of Gastrointestinal Side Effects of Metformin Tablet Compared to Metformin Capsule in Type 2 Diabetes Mellitus Patients.” Journal of Research in Pharmacy Practice vol. 6, 2 (2017): 73-76. doi:10.4103/jrpp.JRPP_17_2. Better treatment options are needed in order to fight back against this metabolic plague.

Signal transduction cascades help to control the millions of individual chemical reactions that occur in the body each second. These reactions occur like a domino chain reaction—as small molecules communicate with each other through covalent and non-covalent interactions to pass along a signal that exerts a biological effect.

FIG. 1 hereof shows the signaling mechanisms in healthy individuals on how the body supports healthy blood-glucose levels. Sugar intake in healthy subjects leads to a rise in blood glucose levels. An intricate process involving the GLP-1, PI3K/AKT, GLUT4, and AMPK signal transduction cascade pathways acts to shuttle glucose out of the bloodstream, into the cells, and produce ATP via glycolysis.

In individuals with metabolic syndrome, signal transduction cascades involved in insulin resistance and formation of advanced glycation products (AGEs) combined with high dietary sugar intake bypass the normal signal transduction cascades and lead to increased insulin levels, increased inflammation, increased glucose concentrations in the bloodstream and, as a result, induction of type 2 diabetes and metabolic syndrome. (See, e.g., FIG. 2.)

Phytonutrients from plants have been used to promote human health via a process known as “poly-pharmacology,” where multiple organic compounds binding with micromolar affinity to multiple enzymatic sites in the body can produce a biological response involved in daily preventative well-care as approached to the mono-pharmacology approached used by the pharmaceutical industry.

### BRIEF SUMMARY

As shown in FIG. 2, many phytonutrients act to maintain the following signal transduction cascades related to maintaining healthy blood-glucose levels in the body: RAGE, NADP oxidase, NLRP, DPP4, GLP-1, PT1B, DAG/PKC, PI3K/AKT, GLUT4, and AMPK.

Described herein is a combination of ingredients capable of regulating STCs involved in blood-sugar control in a subject administered the combination and, as a result, decrease postprandial blood glucose levels, and promote overall metabolic health.

Also described herein are compositions comprising ingredients capable of regulating different signal transduction pathways that help to maintain healthy blood-glucose homeostasis in the body.

Further described is the ability of a unique composition that regulates signal transduction cascades involved in blood-glucose control that demonstrates the ability to lower postprandial glucose levels in a subject in a surprising and unexpected manner relative to a positive control.

Also described is a synergistic combination of signal transduction-regulating phytonutrients/ingredients capable of reducing blood glucose levels both at the peak time of increase after eating (30-50 minutes), while also reducing the total area under the curve in a surprising manner versus a positive control (Gynostemma pentaphyllum).

Particularly described herein are dosage forms for administration to a subject, each dosage form comprising at least four ingredients selected from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus. Typically, such a dosage form will be in a form selected from the group consisting of a softgel, capsule, tablet, gel, powder, gummy, liquid, effervescent, bar, topical patch, serum, lotion, and cream.

Such dosage forms typically contains amount of the selected at least four ingredients to reduce blood glucose in a subject when the subject ingests at least one of the dosage forms on a daily basis. For example, the selected at least four ingredients, when present, are present in the following amounts: from about 200 to about 400 milligrams of okra, from about 25 to about 250 milligrams of quercetin, from about 200 to about 900 milligrams of gypenosides, from about 100 to about 250 milligrams of punicalagins, from about 3 to about 80 milligrams of zinc gluconate, from about 30 to about 100 micrograms of chromium picolinate, from about 30 to about 300 milligrams of curcuminoids, from about 200 to about 1,200 milligrams of cinnamon Ceylon, from about 80 to about 400 milligrams of mangiferin, from about 200 to about 1,250 milligrams of Gymnema sylvestre extract, from about 400 to about 1,300 milligrams of Schisandra chinensis, from about 500 to about 800 milligrams bitter melon, and from about 200 to about 500 milligrams of Costus igneus.

Such a dosage form can further contain at least one other active ingredient to support blood-glucose control that works by mode(s) of action apart from maintenance of signal transduction cascades.

Also described is a method of maintaining a healthy blood sugar level (blood glucose level) in a subject, the method comprising: administering the described dosage form to the subject.

Further described is a method of supporting blood-glucose control in a subject, the method comprising: administering a combination of at least four selected ingredients to the subject, wherein the at least four ingredients are selected are from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus, wherein administration of the composition regulates at least five signal transduction cascades of the subject, wherein the signal transduction pathways are selected from the group consisting of AMPK, Sirt1, PGC-1α, PI3K/AKT, SOD, Nrfs, PGC-1, NF-KB, NLRP3, TNFα, AGE, aldose reductase, PT1B, SIRT, α-glucosidase, AMPK, DPP4, PI3K/AKT, ZIP8, PTP1B, RAGE, GLUT-4, DAG/PKC, AMPK, NLRP3, PI3K/AKT, AMPK, AMPK, PPAR-γ, FGF21, AMPK/Sirt1, JNK, IRS-1, and GLUT-4.

Surprisingly, the methods described herein result, after administration/treatment, in the subject experiencing both lower blood glucose levels at the peak time and lower total area under the curve (“AUC”).

While not intending to be bound by theory, regulation of at least five signal transduction cascades of the subject unexpectedly results in a synergy with respect to glucose regulation and control in the subject.

Utilizing such therapy, the subject typically ingests the selected at least four ingredients (when selected) in the following amounts on a daily basis: from about 200 to about 400 milligrams of okra, from about 25 to about 250 milligrams of quercetin, from about 200 to about 900 milligrams of gypenosides, from about 100 to about 250 milligrams of punicalagins, from about 3 to about 80 milligrams of zinc gluconate, from about 30 to about 100 micrograms of chromium picolinate, from about 30 to about 300 milligrams of curcuminoids, from about 200 to about 1,200 milligrams of Ceylon cinnamon, from about 80 to about 400 milligrams of mangiferin, from about 200 to about 1,250 milligrams of Gymnema sylvestre extract, from about 400 to about 1,300 milligrams of Schisandra chinensis, from about 500 to about 800 milligrams bitter melon, and from about 200 to about 500 milligrams of Costus igneus. A preferred amount of each ingredient is typically within 10% of the middle of the ingredient's respective range.

Also described is such dosage forms for use in reducing blood sugar in a, for example, mammalian subject. This aspect can be especially useful when the subject has been diagnosed as suffering from (or is at risk of suffering from) obesity and/or metabolic syndrome.

Further described herein is a method of making the dosage form comprising admixing the selected ingredients and associating them together into or with the dosage form. The dosage form and method can further include an active or inactive ingredient and/or pharmaceutical excipient into the admixture.

In certain embodiments, the selected phytonutrients may be administered in two different dosage forms, each containing fewer than four of the selected ingredients, but intended to be co-administered to the subject in need thereof so that the at least four ingredients are administered to the subject.

Such dosage forms can be made by admixing the selected phytonutrients and ingredients and associating them together into or with the dosage form.

The dosage form is preferably orally administered to the subject, but other administration modalities are contemplated. When administered orally, the subject ingests the selected at least four phytonutrients.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a flow chart (prior art) depicting the regulation of healthy blood glucose levels in humans via signal transduction cascades.

FIG. 2 is a flow chart depicting the mis-regulation of signal transduction cascades leading to diabetes and metabolic syndrome along with natural molecules from plants that can inhibit this process.

FIG. 3 is a graph depicting the changes in blood-glucose levels after various phytonutrient combinations. Key: Ctrl—Control blood glucose changes after eating without the phytonutrient combination; v3, v3.9, v5, V6—various phytonutrient combinations selected from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus; GP—Gynostemma pentaphyllum, used as a positive control.

FIG. 4 is a graph depicting the total area under the curve (“AUC”) for each phytonutrient composition. Key: Ctrl—Control blood glucose changes after eating without the phytonutrient combination; v3, v3.9, v5, V6—various phytonutrient combinations from selected from the group consisting of okra, quercetin, gypenosides from G. pentaphyllum, punicalagins from pomegranate, zinc gluconate, chromium picolinate, curcuminoids, cinnamon Ceylon, mangiferin, G. sylvestre extract, S. chinensis, bitter melon, and C. igneus; GP used as a positive control.

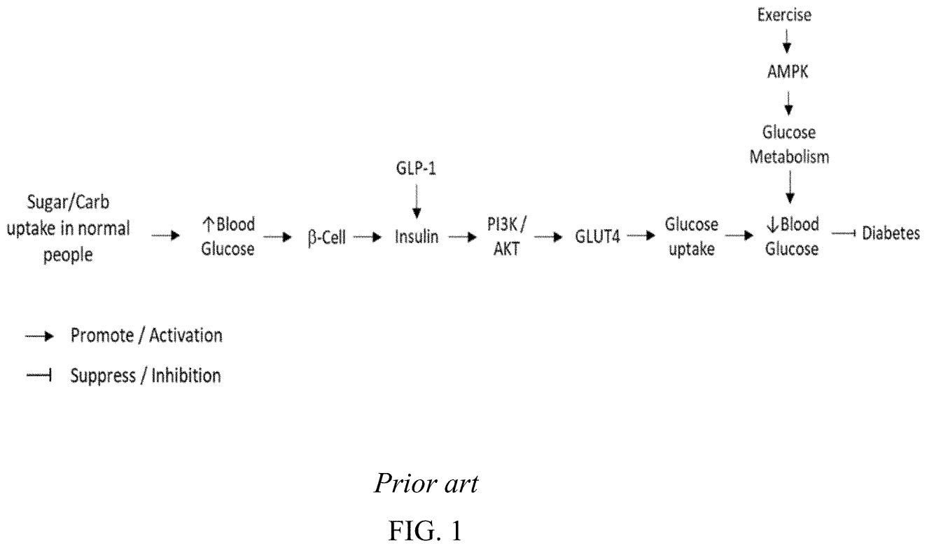

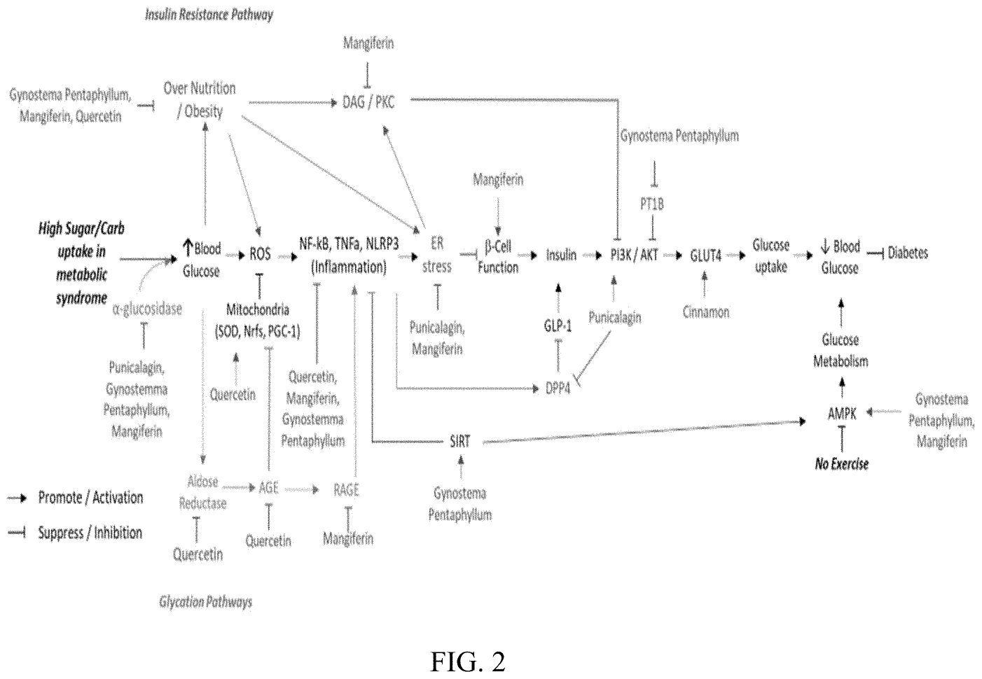

### DETAILED DESCRIPTION

Described are compositions able to lower postprandial blood-glucose levels through regulation of signal transduction cascades (“STCs”).

For purposes of this disclosure, major chemical derivatives of plants fall into three categories, i.e., 1. Macronutrients (e.g., fat, protein, and carbohydrates), 2. Micronutrients (e.g., vitamins and minerals), and 3. Phytonutrients (e.g., organic compounds like polyphenols, sulforaphanes, amines, amides, lactones, ketones, etc.).

Phytonutrients are the “medicinal” component and are commonly a starting point in the drug discovery process.

Phytonutrients described herein have generally been extracted (e.g., from raw plant/fungal material) and/or standardized to the respective compounds for assimilation into the dosage form(s). Such processing increases reproducibility and quality, and reduces the bulk of the selected phytonutrients and allows for smaller, easier to swallow dosage forms.

Although the phytonutrients (and other ingredients) are readily commercially available, processes for extracting phytonutrients are known in the art.

In certain embodiments, the dosage form is utilized or administered together with other inorganic or organic ingredients (such as an anti-diabetic compounds) can be added to the formulation in order to support blood-glucose control that work by modes of action apart from maintenance of signal transduction cascades.

Signal Transduction Cascade Biosignaling Pathways relevant to this disclosure include AMPK, Sirt1, PGC-1α, PI3K/AKT, SOD, Nrfs, PGC-1, NF-KB, NLRP3, TNFα, AGE, aldose reductase, PT1B, SIRT, α-glucosidase, AMPK, DPP4, PI3K/AKT, ZIP8, PTP1B, RAGE, GLUT-4, DAG/PKC, AMPK, NLRP3, PI3K/AKT, AMPK, AMPK, PPAR-γ, FGF21, AMPK/Sirt1, INK, IRS-1, and GLUT-4. The selected signal transduction cascades are related to healthy blood-glucose control in a mammal.

Examples of biosignaling phytonutrients that are useful herein include okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus.

Okra contains phytonutrients, and is readily commercially available. Its components regulate, among other things, the AMPK, Sirt1, PGC-1α, and PI3K/AKT signal transduction pathways. It is said to treat diabetes and improve blood sugar levels. As used herein, a typical daily dosage form (e.g., for use with an adult human subject) contains from about (plus or minus 5% by weight) 200 to about 400 milligrams of okra. The amount can be adjusted for divided doses of the dosage form.

Quercetin is a phytonutrient that is readily commercially available. It regulates, among other things, the SOD, Nrfs, PGC-1, NF-KB, NLRP3, TNFα, AGE, and aldose reductase signal transduction pathways. It is anti-inflammatory, and regulates the cell cycle. As used herein, a typical daily dosage form (e.g., for use with an adult human subject) contains from about (plus or minus 5% by weight) 25 to about 250 milligrams of quercetin. The amount can be adjusted for divided doses of the dosage form.

Gypenosides from Gynostemma pentaphyllum are phytonutrients that are readily commercially available. They regulate, among other things, the PT1B, SIRT, α-glucosidase, and AMPK signal transduction pathways. They are said to offer anti-aging benefits and aid diabetes, high cholesterol, and anxiety. As used herein, a typical daily dosage form (e.g., for use with an adult human subject) contains from about (plus or minus 5% by weight) 200 to about 900 milligrams of Gypenosides. The amount can be adjusted for divided doses of the dosage form.

Punicalagins from pomegranate are phytonutrients that are readily commercially available. They regulate, among other things, the DPP4 and PI3K/AKT signal transduction pathways. They are said to have anti-fungal activity. As used herein, a typical daily dosage form (e.g., for use with an adult human subject) contains from about (plus or minus 5% by weight) 100 to about 250 milligrams of Punicalagins. The amount can be adjusted for divided doses of the dosage form.

Zinc gluconate is readily commercially available. It regulates, among other things, the ZIP8 signal transduction pathway. Zinc is used for the growth, development, and health of body tissues. Zinc gluconate is used to treat and to prevent zinc deficiency. As used herein, a typical daily dosage form (e.g., for use with an adult human subject) contains from about (plus or minus 5% by weight) 3 to about 80 milligrams of zinc gluconate. The amount can be adjusted for divided doses of the dosage form.

Chromium picolinate is readily commercially available. It regulates, among other things, the PTP1B signal transduction pathway. It is said to be effective in improving the body's response to insulin or lowering blood sugar in those with diabetes. It may also help reduce hunger, cravings and binge eating. As used herein, a typical daily dosage form (e.g., for use with an adult human subject) contains from about (plus or minus 5% by weight) 30 to about 110 micrograms of chromium picolinate. The amount can be adjusted for divided doses of the dosage form.

Curcuminoids is a phytonutrient composition that is readily commercially available. It regulates, among other things, the TNF-α signal transduction pathway. It is anti-inflammatory, and regulates cell growth and cell death. As used herein, a typical daily dosage form (e.g., for use with an adult human subject) contains from about (plus or minus 5% by weight) 40 to about 300 milligrams of curcuminoids. The amount can be adjusted for divided doses of the dosage form.

Ceylon cinnamon contains phytonutrients, and is readily commercially available. Its components regulate, among other things, the RAGE, AGE, and GLUT-4 signal transduction pathways. It is said to treat diabetes and improve blood sugar levels. As used herein, a typical daily dosage form (e.g., for use with an adult human subject) contains from about (plus or minus 5% by weight) 400 to about 1,200 milligrams of Ceylon cinnamon. The amount can be adjusted for divided doses of the dosage form.

Mangiferin (from mangos) is a phytonutrient composition that is readily commercially available. It affects, among other things, the DAG/PKC, AMPK, NF-KB, NLRP3, and TNF-α signal transduction pathways. It is has anti-diabetic effects. As used herein, a typical daily dosage form (e.g., for use with an adult human subject) contains from about (plus or minus 5% by weight) 80 to about 400 milligrams of mangiferin. The amount can be adjusted for divided doses of the dosage form.

Gymnema sylvestre extract contains phytonutrients, and is readily commercially available. Its components regulate, among other things, the PI3K/AKT and AMPK signal transduction pathways. It has to treat diabetes, malaria, and snakebites. As used herein, a typical daily dosage form (e.g., for use with an adult human subject) contains from about (plus or minus 5% by weight) 200 to about 1,250 milligrams of G. sylvestre extract. The amount can be adjusted for divided doses of the dosage form.

Schisandra chinensis contains phytonutrients, and is readily commercially available. Its components regulate, among other things, the AMPK and PPAR-γ signal transduction pathways. It is said to prevent early aging and increase lifespan, normalizing blood sugar and blood pressure, stimulating the immune system, and speeding recovery after surgery, and treating liver disease (hepatitis). As used herein, a typical daily dosage form (e.g., for use with an adult human subject) contains from about (plus or minus 5% by weight) 400 to about 1,300 milligrams of S. chinensis. The amount can be adjusted for divided doses of the dosage form.

Bitter melon (or “bitter gourd”) contains phytonutrients, and is readily commercially available. Its components regulate, among other things, the FGF21, AMPK/Sirt1, INK, and NF-KB signal transduction pathways. It is said to lower the body's blood sugar, having properties that act like insulin, which helps bring glucose into the cells for energy. The consumption of bitter melon is thought to assist cells in utilizing glucose and moving it to the body's liver, muscles, and fat. As used herein, a typical daily dosage form (e.g., for use with an adult human subject) contains from about (plus or minus 5% by weight) 500 to about 800 milligrams of bitter melon. The amount can be adjusted for divided doses of the dosage form.

Costus igneus contains phytonutrients, and is readily commercially available. Its components regulate, among other things, the IRS-1 and GLUT-4 signal transduction pathways. It is said to reduce blood sugar levels. As used herein, a typical daily dosage form (e.g., for use with an adult human subject) contains from about (plus or minus 5% by weight) 200 to about 500 milligrams of C. igneus. The amount can be adjusted for divided doses of the dosage form.

Once being apprised of the instant disclosure, a person of ordinary skill in the art will be readily able to make or prepare the dosage forms using Galenical techniques. Preferably, a finished product delivery forms is selected from the group consisting of a softgel, capsule, tablet, gel, powder, gummy, liquid, effervescent, bar, topical patch, serum, lotion, and cream.

The invention is further described with the aid of the following illustrative Examples.

### Example I

The following phytonutrients are thoroughly admixed as close to uniform consistency as possible:

| | | |
|---|---|---|
| Okra | 3 | g |
| Gypenosides | 5 | g |
| Zinc gluconate | 0.400 | g |
| Curcuminoids | 1.7 | g |
| Mangiferin | 2.4 | g |
| Gymnema sylvestre extract | 7.5 | g |
| Schisandra chinensis | 8.5 | g |
| Bitter melon | 6.5 | g |
| Costus igneus | 3.5 | g |

The resulting admixture is divided into 1,000 equal portions each placed into one of 1,000 appropriately-sized hard (or vegan) gelatin capsules with or without a pharmaceutically acceptable diluent.

### Example II

Capsules of EXAMPLE I are administered in a dosing regimen of one capsule twice daily (e.g., with breakfast and dinner) to a subject in order to control blood sugar. The treatment regimen is continued for 13 weeks. The described phytonutrient-based biosignaling supplement supports the subject's health via, for example, controlling blood sugar levels.

### Example III

The following phytonutrients are thoroughly admixed as close to uniform consistency as possible:

| | | |
|---|---|---|
| Quercetin | 1.5 | g |
| Punicalagins | 1.750 | g |
| Zinc gluconate | 0.400 | g |
| Chromium picolinate | 0.700 | mg |
| Curcuminoids | 1.7 | g |
| Mangiferin | 2.4 | g |
| Gymnema sylvestre extract | 7.5 | g |
| Schisandra chinensis | 8.5 | g |

The resulting admixture is divided into 1,000 equal portions each placed into one of 1,000 appropriately-sized hard (or vegan) gelatin capsules with or without a pharmaceutically acceptable diluent.

### Example IV

Capsules of EXAMPLE III are administered in a dosing regimen of one capsule twice daily (e.g., with breakfast and dinner) to a subject in order to control blood sugar. The treatment regimen is continued for 13 weeks. The described phytonutrient-based biosignaling supplement supports the subject's health via, for example, controlling blood sugar levels.

### Example V

The following phytonutrients are thoroughly admixed as close to uniform consistency as possible:

| | | |
|---|---|---|
| Cinnamon Ceylon | 8 | g |
| Mangiferin | 2.4 | g |
| Gymnema sylvestre extract | 7.5 | g |
| Schisandra chinensis | 8.5 | g |
| Bitter melon | 6.5 | g |
| Costus igneus | 3.5 | g |

The resulting admixture is divided into 1,000 equal portions each placed into one of 1,000 appropriately-sized hard (or vegan) gelatin capsules with or without a pharmaceutically acceptable diluent.

### Example VI

Capsules of EXAMPLE V are administered in a dosing regimen of one capsule twice daily to a subject (e.g., together with a subject's breakfast and dinner) in order to control blood sugar. The treatment regimen is continued for 26 weeks. The described phytonutrient-based biosignaling supplement supports the subject's health via, for example, controlling blood sugar levels.

### Example VII

Materials and Methods:

The KETO MOJO™ beta-ketone and blood glucose plus hematocrit and hemoglobin monitoring system TD-4279 kit (Cat #SKU-4011) were used with KETO MOJO™ glucose strips to analyze blood-glucose levels. Peanut butter jelly sandwiches (“PBJ”) were made with whole wheat bread, SKIPPY® creamy peanut butter, and Kroger™ blackberry jelly.

Experiments were performed by taking one PBJ and one banana with or without taking the phytonutrient composition encapsulated in vegetarian capsules. Blood-glucose levels were taken after the first bite of food using the KETO MOJO™ monitor and every 30 minutes thereafter for a total of 3.5 hours. The treatment group took 2 pills of the phytonutrient composition 10 minutes before eating. Sandwiches were made using 1.5 teaspoons of peanut butter and 1.5 teaspoons of jelly between two pieces of whole wheat bread. Blood-glucose concentrations were recorded in mM.

Results and Discussion:

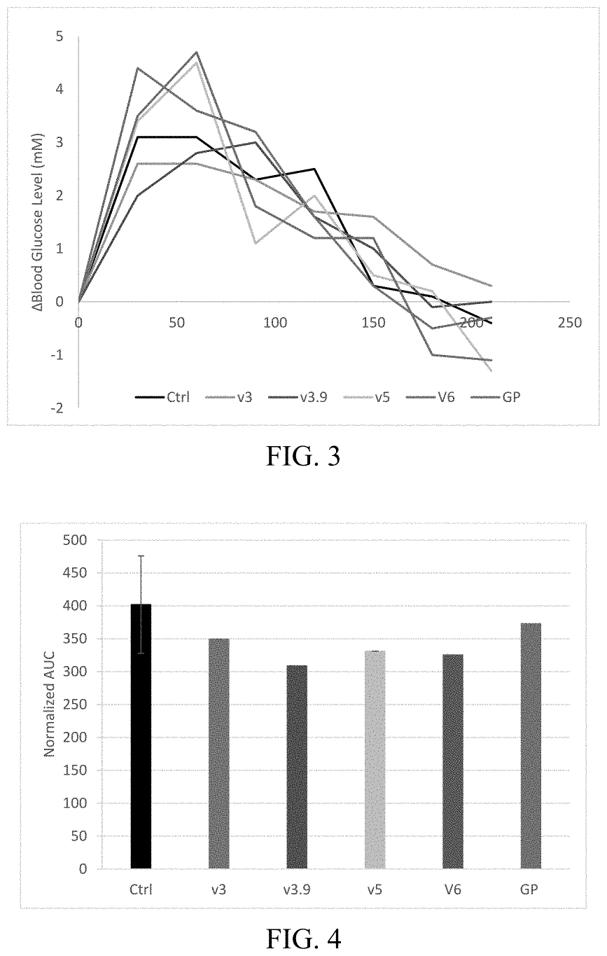

Changes in blood-glucose levels using various combinations of ingredients selected from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus are shown in FIG. 3.

For instance, V3 contained gypenosides, quercetin, chromium picolinate, and punicalagins within the preferred ranges described herein. V3.9 contained gypenosides, quercetin, punicalagin, and cinnamon Ceylon within the preferred ranges described herein. V5 contained gypenosides, quercetin, punicalagin, and mangiferin within the preferred ranges described herein. V6 contained gypenosides, quercetin, punicalagins, mangiferin, cinnamon Ceylon, and chromium picolinate within the preferred ranges described herein. The amount of gypenosides used in the positive control was 600 mg (6.0 g) in the study (taking into account factoring.)

The black line in FIG. 3 shows changes in blood-glucose levels when eating a carbohydrate meal in the absence of any phytonutrients. As shown in FIG. 3, changes in blood-glucose levels peak between 30-50 minutes after consuming food and go back to baseline after three hours. Gynostemma pentaphyllum (“GP”) was used as a positive control, as GP has been shown in the scientific literature to reduce blood glucose and blood-insulin levels. Paralleling what has been seen in the literature, GP (green line) does cause greater changes (i.e., reductions) in blood glucose levels compared to the control-denoting the ability to support healthy blood glucose levels.

Next, different ingredients selected from the group consisting of okra, quercetin, gypenosides from G. pentaphyllum, punicalagins from pomegranate, zinc gluconate, chromium picolinate, curcuminoids, cinnamon Ceylon, mangiferin, G. sylvestre extract, S. chinensis, bitter melon, and C. igneus were assessed for their ability to lower blood glucose levels in the body. While all of the phytonutrient compositions in FIG. 3 lowered blood-glucose levels relative to the control, v3.9, v5, and V6 were the most effective. This data demonstrates the surprising result that phytonutrient compositions regulating signal transduction cascades can lower blood-glucose levels above and beyond a known blood glucose reducer like G. pentaphyllum.

In order to more clearly see the effect of the phytonutrient compositions to support healthy blood glucose levels, the area under the curve (“AUC”) of the control, positive control (i.e., GP), and phytonutrients compositions were calculated and are shown in FIG. 4. FIG. 4 shows the total AUC for each phytonutrient composition. Key: Ctrl—Control blood glucose changes after eating without the phytonutrient combination; v3, v3.9, v5, and V6—various ingredients selected from the group consisting of okra, quercetin, gypenosides from G. pentaphyllum, punicalagins from pomegranate, zinc gluconate, chromium picolinate, curcuminoids, cinnamon Ceylon, mangiferin, G. sylvestre extract, S. chinensis, bitter melon, and C. igneus; GP—used as a positive control. The AUC results in FIG. 4 clearly show that GP reduces blood-glucose levels compared to the positive control while all phytonutrient compositions lowered blood glucose levels relative to GP—with v3.9 being the most effective. AUC reduction for v3.9 is 23.7% compared to the control.

Interestingly and surprisingly, fiber ingredients are known in the scientific literature to reduce blood glucose levels at peak times (30-50 minutes), but fiber ingredients do not decrease AUC—the overall AUC is the same. Rather fiber flattens and lengthens out the glucose curve. In contrast, the described phytonutrient combinations not only decrease glucose concentration at the peak time, but also reduce total AUC. To our knowledge, there are no other phytonutrient compositions reported in the scientific literature that both lower blood glucose levels at the peak time and lower total AUC.

### REFERENCES

(The contents of each of which are incorporated herein by this reference.)
Alam, F., Shafique, Z., Amjad, S. T. & Asad, M. H. H. B. Enzymes inhibitors from natural sources with antidiabetic activity: A review: New targets for antidiabetic treatment. Phytother Res 33, 41-54 (2018).
Aljohi, A., Matou-Nasri, S. & Ahmed, N. Antiglycation and Antioxidant Properties of Momordica charantia. Plos One 11, e0159985 (2016).
An, L. et al. Protective effect of Schisandrae chinensis oil on pancreatic j-cells in diabetic rats. Endocrine 48, 818-25 (2014).
An, X. et al. Punicalagin Protects Diabetic Nephropathy by Inhibiting Pyroptosis Based on TXNIP/NLRP3 Pathway. Nutrients 12, 1516 (2020).
Antony, P. & Vijayan, R. Identification of Novel Aldose Reductase Inhibitors from Spices: A Molecular Docking and Simulation Study. Plos One 10, e0138186 (2015).
Aragno, M. & Mastrocola, R. Dietary Sugars and Endogenous Formation of Advanced Glycation Endproducts: Emerging Mechanisms of Disease. Nutrients 9, 385 (2017).
Ashoush, S., Abou-Gamrah, A., Bayoumy, H. & Othman, N. Chromium picolinate reduces insulin resistance in polycystic ovary syndrome: Randomized controlled trial. J Obstetrics Gynaecol Res 42, 279-85 (2015).
Atrahimovich, D., Samson, A. O., Khattib, A., Vaya, J. & Khatib, S. Punicalagin Decreases Serum Glucose Levels and Increases PON1 Activity and HDL Anti-Inflammatory Values in Balb/c Mice Fed a High-Fat Diet. Oxid Med Cell Longev 2018, U.S. Pat. No. 2,673,076 (2018).
Babu, S. et al. Beta-sitosterol attenuates insulin resistance in adipose tissue via IRS-1/Akt mediated insulin signaling in high fat diet and sucrose induced type-2 diabetic rats. Eur J Pharmacol 873, 173004 (2020).
Benard, O. & Chi, Y. Medicinal Properties of Mangiferin, Structural Features, Derivative Synthesis, Pharmacokinetics and Biological Activities. Mini-rev Med Chem 15, 582-594 (2015).
Cao, H., Polansky, M. M. & Anderson, R. A. Cinnamon extract and polyphenols affect the expression of tristetraprolin, insulin receptor, and glucose transporter 4 in mouse 3T3-L1 adipocytes. Arch Biochem Biophys 459, 214-222 (2007).
Cepas, V., Collino, M., Mayo, J. C. & Sainz, R. M. Redox Signaling and Advanced Glycation Endproducts (AGEs) in Diet-Related Diseases. Antioxidants 9, 142 (2020).
Chai, J.-W., Lim, S.-L., Kanthimathi, M. S. & Kuppusamy, U. R. Gene regulation in β-sitosterol-mediated stimulation of adipogenesis, glucose uptake, and lipid mobilization in rat primary adipocytes. Genes Nutrition 6, 181-8 (2010).
Cortez-Navarrete, M., Martinez-Abundis, E., Perez-Rubio, K. G., Gonzilez-Ortiz, M. & Villar, M. M. Momordica charantia Administration Improves Insulin Secretion in Type 2 Diabetes Mellitus. J Med Food 21, 672-677 (2018).
Davis, P. A. & Yokoyama, W. Cinnamon intake lowers fasting blood glucose: meta-analysis. J Med Food 14, 884-9 (2011).
Du, X.-X. et al. Hypoglycemic Effect of Acidic Polysaccharide from Schisandra chinensis on T2D Rats Induced by High-Fat Diet Combined with STZ. Biol Pharm Bull 42, 1275-1281 (2019).
Du, L. et al. Quercetin inhibited epithelial mesenchymal transition in diabetic rats, high-glucose-cultured lens, and SRA01/04 cells through transforming growth factor-β2/phosphoinositide 3-kinase/Akt pathway. Mol Cell Endocrinol 452, 44-56 (2017).
Fayaz, E., Damirchi, A., Zebardast, N. & Babaei, P. Cinnamon extract combined with high-intensity endurance training alleviates metabolic syndrome via non-canonical WNT signaling. Nutrition Burbank Los Angeles Cty Calif. 65, 173-178 (2019).
Feng, W. et al. Chromium malate alleviates high-glucose and insulin resistance in L6 skeletal muscle cells by regulating glucose uptake and insulin sensitivity signaling pathways. Biometals Int J Role Metal Ions Biology Biochem Medicine 31, 891-908 (2018).
Ganguly, R. et al. Oral chromium picolinate impedes hyperglycemia-induced atherosclerosis and inhibits proatherogenic protein TSP-1 expression in STZ-induced type 1 diabetic ApoE(−/−) mice. Sci Rep-uk 7, 45279 (2017).
Giridharan, V. V. et al. Schisandrin B Ameliorates ICV-Infused Amyloid β Induced Oxidative Stress and Neuronal Dysfunction through Inhibiting RAGE/NF-κB/MAPK and Up-Regulating HSP/Beclin Expression. Plos One 10, e0142483 (2015).
Ge, J., Miao, J.-J., Sun, X.-Y. & Yu, J.-Y. Huangkui capsule, an extract from Abelmoschus manihot (L.) medic, improves diabetic nephropathy via activating peroxisome proliferator-activated receptor (PPAR)-α/γ and attenuating endoplasmic reticulum stress in rats. J Ethnopharmacol 189, 238-49 (2016).
Han, J., Gao, W., Su, D. & Liu, Y. Gypenoside inhibits RANKL-induced osteoclastogenesis by regulating NF-κB, AKT, and MAPK signaling pathways. J Cell Biochem 119, 7310-7318 (2018).
Han, J.-H. et al. Cucurbitane Triterpenoids from the Fruits of Momordica Charantia Improve Insulin Sensitivity and Glucose Homeostasis in Streptozotocin-Induced Diabetic Mice. Mol Nutr Food Res 62, 1700769 (2018).
Hardikar, M. R., Varma, M. E., Kulkarni, A. A., Kulkarni, P. P. & Joshi, B. N. Elucidation of hypoglycemic action and toxicity studies of insulin-like protein from Costus igneus. Phytochemistry 124, 99-107 (2016).
Harris, C. S. et al. Inhibition of advanced glycation end product formation by medicinal plant extracts correlates with phenolic metabolites and antioxidant activity. Planta Med 77, 196-204 (2011).
Hoffman, N. J. et al. Chromium enhances insulin responsiveness via AMPK. J Nutritional Biochem 25, 565-72 (2014).
Huang, H., Chen, G., Dong, Y., Zhu, Y. & Chen, H. Chromium supplementation for adjuvant treatment of type 2 diabetes mellitus: Results from a pooled analysis. Mol Nutr Food Res 62, 1700438 (2017).
Huang, S. et al. The effect of chromium picolinate supplementation on the pancreas and macroangiopathy in type II diabetes mellitus rats. J Diabetes Res 2014, 717219 (2014).
Huyen, V. T. T. et al. Antidiabetic Effects of Add-On Gynostemma pentaphyllum Extract Therapy with Sulfonylureas in Type 2 Diabetic Patients. Evidence-based Complementary Altern Medicine Ecam 2012, 452313 (2012).
Huyen, V. T. T., Phan, D. V., Thang, P., Hoa, N. K. & Ostenson, C. G. Gynostemma pentaphyllum Tea Improves Insulin Sensitivity in Type 2 Diabetic Patients. J Nutrition Metabolism 2013, 1-7 (2013).
Jiang, X., Yu, J., Wang, X., Ge, J. & Li, N. Quercetin improves lipid metabolism via SCAP-SREBP2-LDLr signaling pathway in early stage diabetic nephropathy. Diabetes Metabolic Syndrome Obes Targets Ther 12, 827-839 (2019).
Jin, D. et al. Schisandra polysaccharide increased glucose consumption by up-regulating the expression of GLUT-4. Int J Biol Macromol 87, 555-562 (2016).
Jung, D. Y., Kim, J.-H., Lee, H. & Jung, M. H. Antidiabetic effect of gomisin N via activation of AMP-activated protein kinase. Biochem Bioph Res Co 494, 587-593 (2017).
Karuppagounder, V. et al. Modulation of HMGB1 translocation and RAGE/NFκB cascade by quercetin treatment mitigates atopic dermatitis in NC/Nga transgenic mice. Exp Dermatol 24, 418-423 (2015).
Kiem, P. V. et al. The anti-glycative potentials of pregnane glycosides from Gymnema sylvestre. Phytochem Lett 38, 19-24 (2020).
Liao, Z. et al. Polysaccharide from Okra (Abelmoschus esculentus (L.) Moench) Improves Antioxidant Capacity via PI3K/AKT Pathways and Nrf2 Translocation in a Type 2 Diabetes Model. Mol Basel Switz 24, 1906 (2019).
Lan, C.-Y., Chen, S.-Y., Kuo, C.-W., Lu, C.-C. & Yen, G.-C. Quercetin facilitates cell death and chemosensitivity through RAGE/PI3K/AKT/mTOR axis in human pancreatic cancer cells. J Food Drug Anal 27, 887-896 (2019).
Lee, H. S. et al. Gynostemma Pentaphyllum Extract Ameliorates High-Fat Diet-Induced Obesity in C57BL/6N Mice by Upregulating SIRT1. Nutrients 11, 2475 (2019).
Les, F., Arbonés-Mainar, J. M., Valero, M. S. & López, V. Pomegranate polyphenols and urolithin A inhibit α-glucosidase, dipeptidyl peptidase-4, lipase, triglyceride accumulation and adipogenesis related genes in 3T3-L1 adipocyte-like cells. J Ethnopharmacol 220, 67-74 (2018).
Li, Y. et al. Gymnemic Acid Ameliorates Hyperglycemia through PI3K/AKT- and AMPK-Mediated Signaling Pathways in Type 2 Diabetes Mellitus Rats. J Agr Food Chem 67, 13051-13060 (2019).
Li, X., Zheng, T., Sang, S. & Lv, L. Quercetin Inhibits Advanced Glycation End Product Formation by Trapping Methylglyoxal and Glyoxal. J Agr Food Chem 62, 12152-12158 (2014).
Liao, Z. et al. The anti-nephritic activity of a polysaccharide from okra (Abelmoschus esculentus (L.) Moench) via modulation of AMPK-Sirt1-PGC-1α signaling axis mediated anti-oxidative in type 2 diabetes model mice. Int J Biol Macromol 140, 568-576 (2019).
Megalli et al. Anti-hyperlipidemic and hypoglycemic effects of Gynostemma pentaphyllum in the Zucker fatty rat. J Pharm Pharmaceut Sci 3, 281-291 (2006).
Muthenna et al. Effect of cinnamon and its procyanidin-B2 enriched fraction on diabetic nephropathy in rats. Chem-biol Interact 222, 68-76 (2014). Na, L. et al. Mangiferin supplementation improves serum lipid profiles in overweight patients with hyperlipidemia: a double-blind randomized controlled trial. Sci Rep-uk 5, 10344 (2015).
Nguyen, P. H. et al. New dammarane-type glucosides as potential activators of AMP-activated protein kinase (AMPK) from Gynostemma pentaphyllum. Bioorgan Med Chem 19, 6254-60 (2011).
Oliveira, M. R. de et al. Quercetin and the mitochondria: A mechanistic view. Biotechnol Adv 34, 532-549 (2016).
Pal et al. Mangiferin Attenuates Diabetic Nephropathy by Inhibiting Oxidative Stress Mediated Signaling Cascade, TNFα Related and Mitochondrial Dependent Apoptotic Pathways in Streptozotocin-Induced Diabetic Rats. Plos One 9, e107220 (2014).
Paiva, A. N. et al. Beneficial effects of oral chromium picolinate supplementation on glycemic control in patients with type 2 diabetes: A randomized clinical study. J Trace Elem Med Bio 32, 66-72 (2015).
Peng, X. et al. Beneficial Effects of Cinnamon Proanthocyanidins on the Formation of Specific Advanced Glycation Endproducts and Methylglyoxal-Induced Impairment on Glucose Consumption. J Agr Food Chem 58, 6692-6696 (2010).
Ponnulakshmi, R., Shyamaladevi, B., Vijayalakshmi, P. & Selvaraj, J. In silico and in vivo analysis to identify the antidiabetic activity of beta sitosterol in adipose tissue of high fat diet and sucrose induced type-2 diabetic experimental rats. Toxicol Mech Method 29, 276-290 (2019).
Poornima, B. et al. Advanced glycation end-products inhibitors isolated from Schisandra grandiflora. Nat Prod Res 30, 493-496 (2015).
Rahman, I. U., Khan, R. U., Rahman, K. U. & Bashir, M. Lower hypoglycemic but higher antiatherogenic effects of bitter melon than glibenclamide in type 2 diabetic patients. Nutr J 14, 13 (2015).
Rayamajhi, N. et al. Quercetin induces mitochondrial biogenesis through activation of HO-1 in HepG2 cells. Oxid Med Cell Longev 2013, 154279 (2013).
Sheng, Z. et al. Capability of Polygonum cuspidatum extract in inhibiting AGEs and preventing diabetes. Food Sci Nutrition 7, 2006-2016 (2019).
Shi, Z.-L. et al. In Vitro and In Vivo Effects of Norathyriol and Mangiferin on α-Glucosidase. Biochem Res Int 2017, U.S. Pat. No. 1,206,015 (2017).
Sadowska-Bartosz, I. & Bartosz, G. Prevention of Protein Glycation by Natural Compounds. Molecules 20, 3309-3334 (2015).
Suchal, K. et al. Protective effect of mangiferin on myocardial ischemia-reperfusion injury in streptozotocin-induced diabetic rats: role of AGE-RAGE/MAPK pathways. Sci Rep-uk 7, 42027 (2017).
Tian, H. et al. Chromium picolinate supplementation for overweight or obese adults. Cochrane Database Syst Rev 11, CD010063 (2013).
Vistoli, G. et al. Advanced glycoxidation and lipoxidation end products (AGEs and ALEs): an overview of their mechanisms of formation. Free Radical Res 47, 3-27 (2013).
Wang, Y. & Yao, M. Effects of chromium picolinate on glucose uptake in insulin-resistant 3T3-L1 adipocytes involve activation of p38 MAPK. J Nutritional Biochem 20, 982-91 (2009).
Wang, Z. et al. Antioxidant and anti-inflammatory activities of an anti-diabetic polysaccharide extracted from Gynostemma pentaphyllum herb. Int J Biol Macromol 145, 484-491 (2019).
Wang, Z. Q. et al. Bioactives from bitter melon enhance insulin signaling and modulate acyl carnitine content in skeletal muscle in high-fat diet-fed mice. J Nutritional Biochem 22, 1064-1073 (2011).
Wang, Z. et al. Anti-diabetic activity evaluation of a polysaccharide extracted from Gynostemma pentaphyllum. Int J Biol Macromol 126, 209-214 (2019).
Xia, X. et al. Jiaogulan tea (Gynostemma pentaphyllum) potentiates the antidiabetic effect of white tea via the AMPK and PI3K pathways in C57BL/6 mice. Food Funct 11, 4339-4355 (2020).
Xu, X. et al. Mangiferin suppresses endoplasmic reticulum stress in perivascular adipose tissue and prevents insulin resistance in the endothelium. Eur J Nutr 57, 1563-1575 (2018).
Xu et al. Dammaranes from Gynostemma pentaphyllum and synthesis of their derivatives as inhibitors of protein tyrosine phosphatase 1B. Bioorgan Med Chem 18, 3934-9 (2010).
Yang, S. J. et al. Preventive effects of bitter melon (Momordica charantia) against insulin resistance and diabetes are associated with the inhibition of NF-κB and JNK pathways in high-fat-fed OLETF rats. J Nutritional Biochem 26, 234-40 (2014).
Yin, R. V. & Phung, O. J. Effect of chromium supplementation on glycated hemoglobin and fasting plasma glucose in patients with diabetes mellitus. Nutr J 14, 14 (2015).
Yoon, N. A. et al. Anti-obesity Activity of Ethanol Extract from Bitter Melon in Mice Fed High-Fat Diet. Dev Reproduction 23, 129-138 (2019).
Yu et al. Bitter melon extract attenuating hepatic steatosis may be mediated by FGF21 and AMPK/Sirt1 signaling in mice. Sci Rep-uk 3, 3142 (2013).
Zhang, X.-S. et al. Protein tyrosine phosphatase 1B inhibitory effect by dammarane-type triterpenes from hydrolyzate of total Gynostemma pentaphyllum saponins. Bioorg Med Chem Lett 23, 297-300 (2012).
Zhang, H. et al. Gypenosides improve diabetic cardiomyopathy by inhibiting ROS-mediated NLRP 3 inflammasome activation. J Cell Mol Med 22, 4437-4448 (2018).
Zhang, L. et al. Insights into the Mechanism of Quercetin against BSA-Fructose Glycation by Spectroscopy and High-Resolution Mass Spectrometry: Effect on Physicochemical Properties. J Agr Food Chem 67, 236-246 (2019).
Zhong, J., Reece, E. A. & Yang, P. Punicalagin exerts protective effect against high glucose-induced cellular stress and neural tube defects. Biochem Bioph Res Co 467, 179-84 (2015).

What is claimed is:

1. A dosage form for administration to a subject, the dosage form comprising at least four ingredients selected from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus.

2. The dosage form of claim 1, wherein the dosage form contains amounts of the selected at least four ingredients to reduce blood glucose in a subject when the subject ingests at least one of the dosage forms on a daily basis.

3. The dosage form of claim 1, wherein the selected at least four ingredients, when present, are present in the following amounts:
from about 200 to about 400 milligrams of okra,
from about 25 to about 250 milligrams of quercetin,
from about 200 to about 900 milligrams of gypenosides,
from about 100 to about 250 milligrams of punicalagins,
from about 3 to about 80 milligrams of zinc gluconate,
from about 30 to about 100 micrograms of chromium picolinate,
from about 30 to about 300 milligrams of curcuminoids,
from about 200 to about 1,200 milligrams of cinnamon Ceylon,
from about 80 to about 400 milligrams of mangiferin,
from about 200 to about 1,250 milligrams of Gymnema sylvestre extract,
from about 400 to about 1,300 milligrams of Schisandra chinensis,
from about 500 to about 800 milligrams bitter melon, and
from about 200 to about 500 milligrams of Costus igneus.

4. The dosage form of claim 1, wherein the dosage form is selected from the group consisting of a softgel, capsule, tablet, gel, powder, gummy, liquid, effervescent, bar, topical patch, serum, lotion, and cream.

5. The dosage form of claim 1, further comprising:
at least one other active ingredient to support blood-glucose control that works by mode(s) of action other than maintenance of signal transduction cascades in a subject.

6. A method of maintaining a healthy blood sugar level in a subject, the method comprising:
administering the dosage form of claim 2 to the subject.

7. A method of support blood-glucose control in a subject, the method comprising:
administering a combination of at least four selected ingredients to the subject, wherein the at least four ingredients are selected are from the group consisting of okra, quercetin, gypenosides from Gynostemma pentaphyllum, punicalagins from pomegranate, zinc gluconate, chromium picolinate, curcuminoids, cinnamon Ceylon, mangiferin, Gymnema sylvestre extract, Schisandra chinensis, bitter melon, and Costus igneus,
wherein administration of the composition regulates at least five signal transduction cascades of the subject, wherein the signal transduction pathways are selected from the group consisting of AMPK, Sirt1, PGC-1α, PI3K/AKT, SOD, Nrfs, PGC-1, NF-KB, NLRP3, TNFα, AGE, aldose reductase, PT1B, SIRT, α-glucosidase, AMPK, DPP4, PI3K/AKT, ZIP8, PTP1B, RAGE, GLUT-4, DAG/PKC, AMPK, NLRP3, PI3K/AKT, AMPK, AMPK, PPAR-γ, FGF21, AMPK/Sirt1, JNK, IRS-1, and GLUT-4.

8. The method according to claim 7, wherein the subject ingests the selected at least four ingredients, when selected, in the following amounts on a daily basis:
from about 200 to about 400 milligrams of okra,
from about 25 to about 250 milligrams of quercetin,
from about 200 to about 900 milligrams of gypenosides,
from about 100 to about 250 milligrams of punicalagins,
from about 3 to about 80 milligrams of zinc gluconate,
from about 30 to about 100 micrograms of chromium picolinate,
from about 30 to about 300 milligrams of curcuminoids,
from about 200 to about 1,200 milligrams of Ceylon cinnamon,
from about 80 to about 400 milligrams of mangiferin,
from about 200 to about 1,250 milligrams of Gymnema sylvestre extract,
from about 400 to about 1,300 milligrams of Schisandra chinensis,
from about 500 to about 800 milligrams bitter melon, and
from about 200 to about 500 milligrams of Costus igneus.

9. A method of reducing blood glucose levels in a mammalian subject, the method comprising:
administering to the subject the dosage form of claim 2.

10. A method of making the dosage form of claim 1, the method comprising admixing the selected ingredients and associating them together into or with the dosage form.

11. The method according to claim 10, further comprising including an inactive ingredient and/or pharmaceutical excipient into the admixture.

12. The method according to claim 6, wherein the subject is suffering from obesity and/or metabolic syndrome.

13. The method according to claim 6, wherein after administration, the subject experiences both lower blood glucose levels at the peak time and lower total area under the curve (“AUC”).
